(19) United States
(12) Patent Application Publication
(10) Pub. No.: US 2008/0040831 A1
(43) Pub. Date: Feb. 21, 2008

(54) Temperature altering garment and methods of use thereof

(76) Inventors: Ali Nilforushan, Cardiff-by-the-Sea, CA (US); Kevin Bello, Escondido, CA (US)

Correspondence Address: GORDON & REES LLP, 101 WEST BROADWAY, SUITE 1600, SAN DIEGO, CA, 92101, US

(21) Appl. No.: 11/804,552

(22) Filed: May 18, 2007

Related U.S. Application Data
(60) Provisional application No. 60/838,666, filed on Aug. 17, 2006.

(51) Int. Cl. A41D 1/00; A61F 7/00; F25D 23/12; H05B 1/00

(52) U.S. Cl. 2/69; 219/211; 607/96; 62/259.3

(57) ABSTRACT

A garment is provided including an article of clothing to which one or more thermal transfer elements are removably and adjustably secured by the engagement of a first fastener with a second fastener. The character of the first and second fastener allow the location of the thermal transfer element to be adjusted multiple times without significantly weakening the bond between the fasteners. Heat is transferred between the thermal transfer element and the selected body part of the wearer, while the wearer's hands remain unoccupied so that the wearer can participate in other activities while receiving a heat transfer treatment.

### CROSS-REFERENCE TO RELATED APPLICATION

The present application claims the benefit of Provisional Application No. 60/838,666 which was filed on Aug. 17, 2006.

### FIELD OF THE INVENTION

This invention relates generally to clothing that transfers heat to or from the body of the wearer, and is particularly concerned with a garment for applying hot and/or cold thermal packs to the body of the wearer, and methods of use thereof.

The reasons for heating or cooling parts of the human body are well known and include, without limitation, prevention of injuries, treatment of injuries, pain relief, improving flexibility, warming a person in a cold environment, and cooling a person in a hot environment. A common method for heating or cooling a body part of a person is to place it in contact with a hot or cold object. Contacting a hot object with a body part transfers heat from the object to the body part; contacting a cold object with a body part transfers heat from the body part to the object. A common object used for this purpose is a thermal pack, which may be a hot pack or cold pack.

There are many well known methods for applying thermal packs to human body parts. One method is to manually hold a thermal pack in contact with a body part that is intended to be heated or cooled. This method has significant disadvantages. First, a person must engage the assistance of another in order to apply a thermal pack to a hard-to-reach area. Additionally, a person cannot alone simultaneously apply thermal packs to more than a few areas on the body, since a person is only able to use two hands to hold the packs in place. Finally, because the hands of a person using this method will be occupied, the person will be unable to simultaneously engage in other activities that require use of the hands.

Another method for applying a thermal pack to a body part is to physically attach it. In this method the person uses straps, wraps, adhesives, or other means to secure a thermal pack to a body part. This method presents several disadvantages. First, the person's movement may be restricted by attachment of thermal packs to certain commonly treated parts, such as the knee. Additionally, it may be cumbersome to temporarily hold the pack in place with one hand while simultaneously trying to use a strap or other means to secure the pack in place. Furthermore, attaching a thermal pack too tightly may impede circulation and cause tissue damage by disrupting blood flow through the treated body part. Finally, it is difficult to physically attach a thermal pack to certain body parts, such as the back, without engaging the assistance of another.

Thus, although there are known methods for applying a thermal pack to a human body part, each of these methods suffers from problems. Information relevant to attempts to address these problems can be found in U.S. Pat. No. 5,038,779 issued to Barry et al., U.S. Pat. No. 5,826,273 issued to Eckes, and U.S. Pat. No. 7,065,983 issued to Trinh et al. However, each of these references suffers from significant disadvantages including, without limitation, one or more of the following: the thermal pack can only be attached to certain body parts, the location of the thermal pack cannot be adjusted at all, the location of the thermal pack cannot be adjusted with much accuracy or precision, or the means for attaching the thermal pack to the body part can only be used once or wears out with multiple uses.

Accordingly, there is a need for a device that does not suffer from any of the aforementioned disadvantages; there is a need for a device capable of removably and adjustably applying thermal packs to specifically targeted parts of a person's body, leaving that person's hands free to simultaneously engage in other activities.

### SUMMARY OF THE INVENTION

The present invention is directed to a garment that satisfies this need, and methods of use thereof. According to one embodiment of the present invention, a garment for applying a thermal transfer element to a part of the body of a person is provided. The garment includes an article of clothing having a first layer of flexible material. Attachable to the article of clothing is a thermal transfer element. One fastener is located on the article of clothing, and another fastener is located on the thermal transfer element, whereby engagement of the first fastener and the second fastener removably and adjustably secures the thermal transfer element to the article of clothing. As used herein, the term “garment” shall refer to the combination of the article of clothing and thermal transfer element; the term “article of clothing” shall only refer to the piece of apparel to which the thermal transfer element is secured. The thermal transfer element is any object suitable for transferring heat to or from the wearer's body. The location of the thermal transfer element is adjustable by disengaging the first fastener from the second fastener, placing the thermal transfer element in an alternate location, and reengaging the two fasteners.

In one embodiment of the present invention, the first fastener is loop material and the second fastener is hook material. In another embodiment, the first fastener is hook material and the second fastener is loop material.

In one embodiment, the first fastener is found on the interior surface of the first layer of flexible material. In one embodiment, the first fastener covers substantially this entire surface. In one embodiment, substantially this entire surface comprises loop material. In another embodiment, substantially this entire surface comprises hook material.

In one embodiment, the first fastener is found on the exterior surface of the first layer of flexible material. In one embodiment, the first fastener covers substantially this entire surface. In one embodiment, substantially this entire surface comprises loop material. In another embodiment, substantially this entire surface comprises hook material.

In one embodiment of the present invention, the thermal transfer element comprises a thermal pack. In another embodiment, the thermal transfer element comprises a thermal pack and a pouch that holds the thermal pack, the second fastener being located on the pouch. In one embodiment, the thermal transfer element comprises an inflatable air bladder.

In one embodiment of the present invention, the article of clothing is selected from the group consisting of vest, short-sleeve shirt, long-sleeve shirt, sleeve, shorts, pants, legging, bodysuit, skirt, dress, gown, robe, glove, sock, and hat.

In one embodiment of the present invention, the article of clothing snugly fits the body of the wearer such that the article of clothing presses the thermal transfer element against a part of the body of the wearer.

In one embodiment of the present invention, the article of clothing further comprises an outer layer of flexible material. In one embodiment, the first fastener is located on the interior surface of this layer. In another embodiment, this surface comprises the first fastener, which may be hook material or loop material.

In one embodiment of the present invention, the first fastener is located on the exterior surface of the outer layer of flexible material. In another embodiment, this surface comprises the first fastener, which may be hook material or loop material.

In another embodiment of the present invention, the article of clothing comprises a detachable segment such that the rest of the article of clothing may be worn without the detached segment, and the detached segment may be worn without the rest of the article of clothing. In one embodiment, the detachable segment comprises the first fastener.

In another embodiment of the present invention, a garment includes an article of clothing and a thermal transfer element that is held in place between the article of clothing and a part of the body of a person by forces generated by the article of clothing. In one embodiment these forces are augmented by a snugly fitting article of clothing. In another embodiment the article of clothing further comprises tightenable straps or cords whose whereby the force generated by the article of clothing is variable. In another embodiment the article of clothing further comprises an air bladder that may be inflated to increase the force generated by the article of clothing. In another embodiment, the thermal transfer element further comprises an air bladder that may be inflated to increase the force.

In yet another embodiment of the present invention, the article of clothing comprises a moisture absorbent material. In another embodiment, the article of clothing comprises a thermoconductive material. In another embodiment, the article of clothing comprises a thermoreflective material.

Another embodiment of the present invention relates to a method of transferring heat between a thermal pack and a human body part. First, a body part with which to transfer heat is selected. A thermal pack is brought to a desired temperature, and an article of clothing having a first fastener is put on a person. Using an engagement between a first fastener and a second fastener, the thermal pack is removably and adjustably secured to a location on the article of clothing that contacts the selected body part when the article of clothing is worn. Once attached, heat is transferred between the thermal pack and the selected body part. In one embodiment the first fastener is loop material and the second fastener is hook material. In another embodiment, the first fastener is hook material and the second fastener is loop material. In another embodiment, the thermal pack is placed in a pouch having a second fastener.

Yet another embodiment of the present invention relates to a method for heating or cooling the body of an athlete while the athlete is engaged in an athletic activity. A body part to heat or cool is selected, and an article of clothing is put on the body of an athlete, the article of clothing at least partially covering the selected body part. A location on the article of clothing that contacts the selected body part when the article of clothing is worn is selected, and a thermal pack is brought to a desired temperature. The thermal pack is removably and adjustably secured to the selected location, and the selected body part is heated or cooled while the athlete is engaged in an athletic activity. In another embodiment, the thermal pack is placed in a pouch. In another embodiment, the thermal pack is removably and adjustably secured to the selected location using a hook and loop fastener.

Yet another embodiment of the present invention relates to a method of performing temperature enhanced physical therapy on a patient. A body part of the patient to receive the therapy is selected, and an article of clothing is put on the body of the patient. A location on the article of clothing that contacts the selected body part when the article of clothing is worn by the patient is selected, and a thermal pack is brought to a desired temperature. The thermal pack is secured to the selected location, and heat is transferred between the selected body part and the thermal pack while the patient receives physical therapy on the body part. In one embodiment the thermal pack is removably and adjustably secured to the selected location using a hook and loop fastener. In another embodiment, the thermal pack is placed in a pouch.

Another embodiment of the present invention relates to a method of warming the body of a person in a cold environment. A body part to be warmed is selected, and an article of clothing having a first fastener is put on the body of the person. A location on the article of clothing that contacts the selected body part when the article of clothing is worn is selected, and a thermal pack is brought to a desired temperature The thermal pack is removably and adjustably secured to selected location by engaging the first fastener with a second fastener, thus permitting heat to transfer to the selected body part while the person is in a cold environment. In another embodiment, the thermal pack is placed in a pouch having a second fastener. In one embodiment, the first fastener is loop material and the second fastener is hook material. In another embodiment the first fastener is hook material and the second fastener is loop material.

Yet another embodiment of the present invention relates to a method of pressing a thermal pack against a body part of a person to increase the rate of heat transfer between the body part and the thermal pack. A body part with which to transfer heat is selected, and a snugly fitting article of clothing is put on the body of a person. A thermal pack is brought to a desired temperature, and a location on any surface of the article of clothing, other than the outermost surface, that contacts the selected body part when the article of clothing is worn is selected. The thermal pack is secured to the selected location, and the snug fit of the article of clothing presses the thermal pack against the selected body part, so that heat transfers between the thermal pack and the selected body part. In one embodiment the thermal pack is secured to the selected location using a hook and loop fastener. In another embodiment the thermal pack is placed in a pouch.

Yet another embodiment of the present invention relates to a method of simultaneously heating one body part while cooling another body part. A first body part to be heated, and a second body part to be cooled are selected. A first thermal pack is heated, and a second thermal pack is cooled. An article of clothing having a first fastener and a second fastener is put on the body of a person. A first location on the article of clothing that contacts the selected first body part to be heated when the article of clothing is worn is selected. A second location on the article of clothing that contacts the selected second body part to be cooled when the article of clothing is worn is also selected. The first thermal pack is removably and adjustably secured to the selected first location by engaging the first fastener with a third fastener. The second thermal pack is removably and adjustably secured to the selected second location by engaging the second fastener with a fourth fastener. Heat then transfers between the thermal packs and the selected body parts. In one embodiment, the thermal packs are placed in pouches. In another embodiment, the first and second fasteners are loop material and the third and fourth fasteners are hook material.

There are many advantages to the various embodiments of the present invention. An advantage of one embodiment is the ability to adjustably secure a thermal transfer element to any body part covered by the garment. In this embodiment, the location of the thermal transfer element is adjustable over substantially an entire surface of an article of clothing because the fastener on the article of clothing is not fixed in a single location. Instead, in this embodiment, the fastener substantially covers an entire surface of the article of clothing. Thus a user of this embodiment of the present invention can precisely and adjustably target any body part covered by the article of clothing, instead of only specific, unchangeable locations.

Another advantage of an embodiment of the present invention is the ability to adjustably secure a thermal transfer element to an article of clothing multiple times without weakening the strength of the engagement between the fasteners. Other systems, which use adhesives to secure thermal transfer elements to an article of clothing, do not have this advantage because the adhesive weakens after every adhesion. Eventually, after multiple adhesions, the adhesive will no longer hold the thermal transfer element to the article of clothing. The engagement between fasteners of an embodiment of the present invention, however, does not so weaken due to repeated use.

Another advantage of an embodiment of the present invention is that it allows a person to apply a thermal transfer element to a hard to reach area, such as the back, without requiring the assistance of another. The person may simply secure the thermal transfer element to the back of the article of clothing before putting on the article of clothing. In other systems, it is very awkward or cumbersome to apply a thermal transfer element to the back without the assistance of another.

Another advantage of an embodiment of the present invention is that it allows a user to apply a thermal transfer element to a body part without occupying the hands. Thus, the user may engage in activities that require the use of one or both hands while a thermal transfer element is held against a body part. A further, related advantage is the ability to hold multiple thermal transfer elements, including both hot and cold thermal transfer elements simultaneously, to multiple body parts. Manual systems are limited by the number of thermal transfer elements the person can hold in the hands. The present invention, however, is not so limited.

A further related advantage of an embodiment of the present invention is the ability to engage in an athletic activity while a thermal transfer element is held against a body part. An athlete wearing a garment of the present invention is able to keep body parts warm during periods of inactivity, and need not remove the garment prior to resuming the athletic activity. Wearing the garment during the athletic activity may also prevent injury to muscles by keeping them warm. Alternatively, an athlete may cool parts of the body during strenuous activity to prevent overheating. Furthermore, a thermal transfer element secured to a body part may act as padding for that body part. Finally, after the athletic activity, the garment can be used for treatment of sore muscles.

Yet another advantage of an embodiment of the present invention is the ability to press a thermal transfer element against a body part. When the article of clothing snugly fits the body of the wearer, and when the thermal transfer element is secured inside the article of clothing, the snug fit presses the thermal transfer element against the body part. This pressure not only increases the efficiency of heat transfer, but also helps prevent swelling of an injured body part when a cold pack is applied under pressure.

### BRIEF DESCRIPTION OF THE DRAWINGS

FIG. 1 is a front perspective view of a garment comprising an article of clothing having an inner layer and an outer layer, with thermal transfer elements inside the outer layer indicated with dashed lines, according to a first embodiment of the invention.

FIG. 2 is a back perspective view of the garment of FIG. 1.

FIG. 3 is a front elevation view of the article of clothing of the garment of FIG. 1.

FIG. 4 is a side elevation view of the article of clothing of the garment of FIG. 1.

FIG. 5 is a front elevation view of a thermal transfer element according to one embodiment of the invention.

FIG. 6 is a back elevation view of the thermal transfer element of FIG. 5.

FIG. 7 is a front elevation view of a thermal pack according to one embodiment of the invention.

FIG. 8 is a side perspective view of a thermal transfer element according to one embodiment of the invention, before and after a thermal pack is inserted into a pouch.

FIG. 9 is a front perspective view of a garment comprising an article of clothing having only a first layer, with hidden secured thermal transfer elements indicated with dashed lines, according to another embodiment of the invention.

FIG. 10 is a side elevation view of the article of clothing of the garment of FIG. 9.

FIG. 11 is a front perspective view of a garment comprising a different article of clothing (namely shorts), according to another embodiment of the invention.

FIG. 12 is a back elevation view of the garment of FIG. 11.

FIG. 13 is a front elevation view of a detachable segment of an article of clothing of a garment according to another embodiment of the invention.

FIG. 14 is front perspective view of the garment of FIG. 13.

FIG. 15 is a front perspective view of the inside of an article of clothing comprising an inflatable air bladder, according to another embodiment of the invention.

FIG. 16 is a front perspective view of the outside of a garment comprising the article of clothing of FIG. 15, shown with the air bladder inflated and thermal transfer elements in place, according to one embodiment of the invention.

FIG. 17 is a back perspective view of the garment of FIG. 16.

FIG. 18 is a side perspective view of a thermal transfer element held in place inside an article of clothing by a strap and buckle, according to another embodiment of the invention.

FIG. 19 is a front perspective view of a garment with the thermal transfer element of FIG. 18 secured inside the article of clothing.

FIG. 20 is a back perspective view of the garment of FIG. 19.

### DETAILED DESCRIPTION

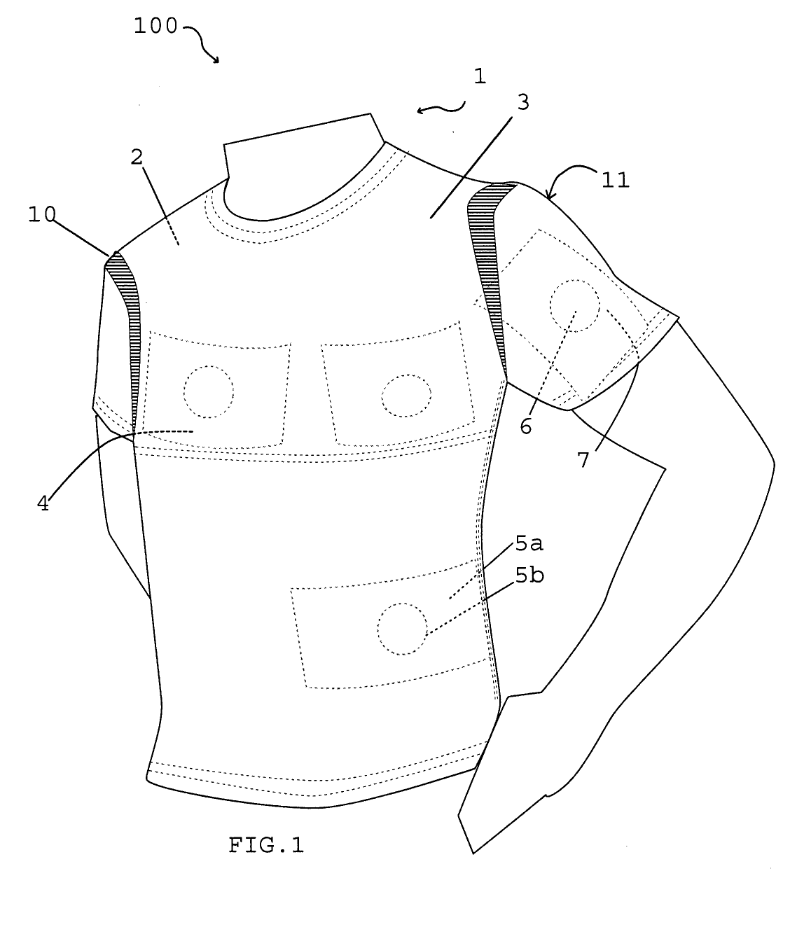

As shown in FIGS. 1 to 3, an exemplary embodiment of the present invention is a garment 100 for applying a thermal transfer element to the body of a human comprising an article of clothing 1 having a first layer 10, an outer layer 11, a thermal transfer element 4, a first fastener 5A, and a second fastener 5B. The garment 100 is the entire combination; the article of clothing 1 is the piece of clothing to which thermal transfer elements are secured.

An engagement between the first fastener 5A and the second fastener 5B removably and adjustably secures the thermal transfer element 4 to any location 6 on the interior surface 2 of the outer layer 11 of the article of clothing 1. “Removably and adjustably” secured means that the thermal transfer element can be removed without damaging the fasteners or otherwise dismantling the garment, and be reattached in a different location. After attachment, heat transfer takes place between the thermal transfer element 4 and a body part 7 in contact with the location 6 on the article of clothing 1 where the thermal transfer element 4 is secured. The location 6 is “in contact” with the body part 7 even if there are one or more layers of material between the location 6 and the body part 7. In other words, it is not necessary for the location 6 to touch the skin of the body part 7 for the location 6 to “contact” the body part 7, so long as they immediately adjoin each other.

As best illustrated in FIGS. 4, 10 and 11, an interior surface 2 of the article of clothing 1 is any surface of any layer of the article of clothing that faces inwardly toward the wearer. An exterior surface 3 of the article of clothing 1 is any surface of any layer of the article of clothing 1 that faces outwardly away from the wearer.

In an exemplary embodiment, as best illustrated in FIG. 4, the article of clothing 1 has a first layer 10 and an outer layer 11 such that there is room for thermal transfer elements 4 between the layers. In an exemplary embodiment, the outer layer 11 is made from a nylon, polyester, and spandex blend, and the interior surface 2 of the outer layer 11 is brushed to create loop material suitable for a hook and loop fastener. The process of brushing is well known in the art, and is essentially a finishing process for materials, particularly knit or woven fabrics, in which brushes or other devices abrade the material to create a looped or napped surface that is suitable for use in a hook and loop fastener. The first layer 10 may be any flexible material but in an exemplary embodiment is a fine mesh material.

The article of clothing 1 may be made from any flexible material. More specifically, and without limitation, the material may be moisture absorbent, breathable, stretchable, meshed, or any blend or combination thereof. Even more particularly, the material may be a blend of nylon, polyester, and spandex. The material may also be thermoconductive in order to better conduct heat between the thermal transfer element 4 and the body part 7. A thermoconductive material is any material that is a good conductor of heat. The material may also be a thermoreflective material, which is any material that is a good insulator. For example, the outer layer 11 of the article of clothing 1 may comprise a thermoreflective material to help keep heat inside the article of clothing 1. The material may further comprise either the first fastener 5A or the second fastener 5B, meaning that these fasteners may be either an integral part of the material or affixed thereto. For example, one or more surfaces of the article of clothing 1 may be brushed such that it comprises loop material suitable for a hook and loop fastener.

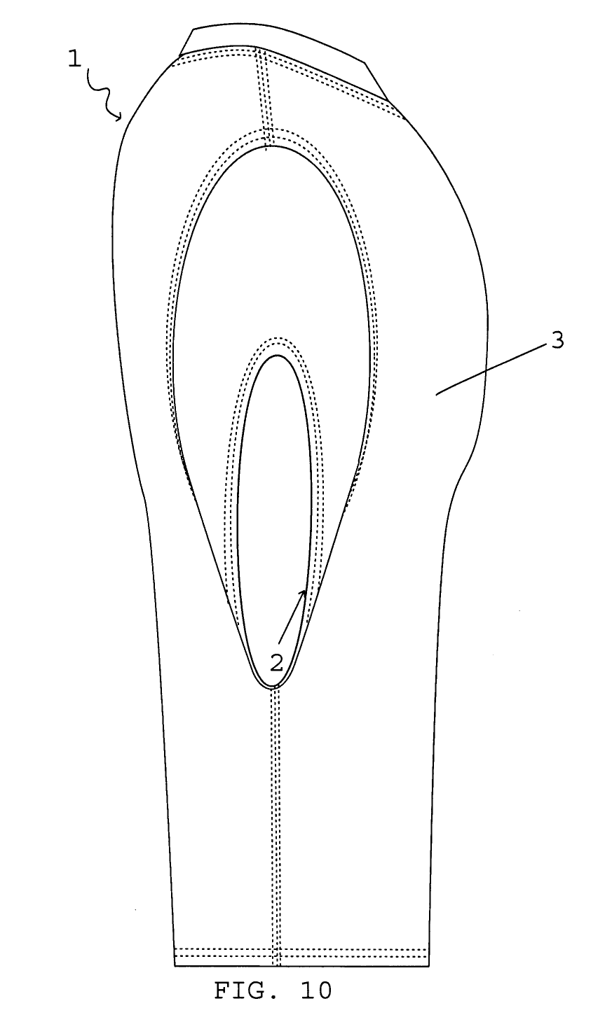

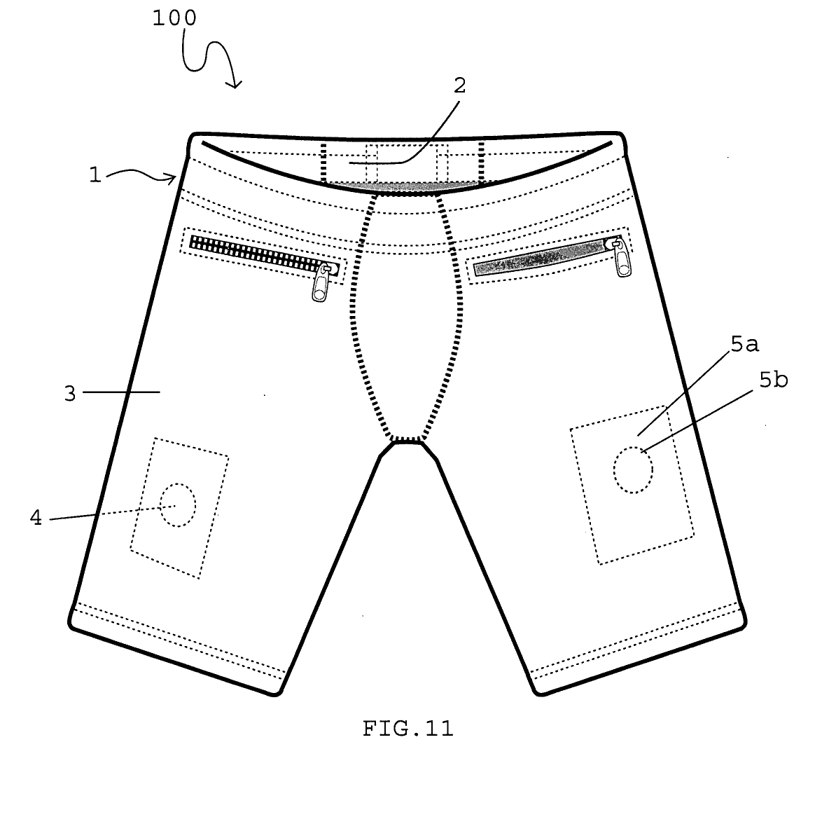

The article of clothing 1 may be of any type including, without limitation, shirts, vests, shorts, pants, bodysuits, skirts, dresses, robes, socks, gloves, or hats. For example, as shown in FIGS. 11 and 12, the article of clothing 1 may be a pair of shorts. It may come in a variety of shapes and sizes to accommodate the body shapes of men, women, and children of all sizes. The article of clothing 1 is worn on the body of a person, and may be worn as any layer of clothing, including as an undergarment or as an over-garment.

In an exemplary embodiment of the present invention, the article of clothing 1 snugly fits the body of the wearer such that any thermal transfer element 4 inside the article of clothing 1 is pressed against the body part 7 due to the snug fit of the article of clothing 1. The phrase “snug fit” means that the article of clothing 1 is tight on the wearer's body, having little or no slack in the material. The phrase “pressed against the body part,” means a force exerted on the thermal transfer element 4 that tends to push it toward the body of the wearer. This pressure increases the efficiency of heat transfer by increasing the surface area of the thermal transfer element 4 in contact with the body part 7, because any gaps between the surface of the thermal transfer element 4 and the body part 7 will be eliminated. Furthermore, to be “pressed against a body part,” it is not necessary for the thermal transfer element 4 to touch the skin of the wearer, so long as the body part 7 provides a reactionary force in opposition to the force exerted by the snugly fitting article of clothing 1.

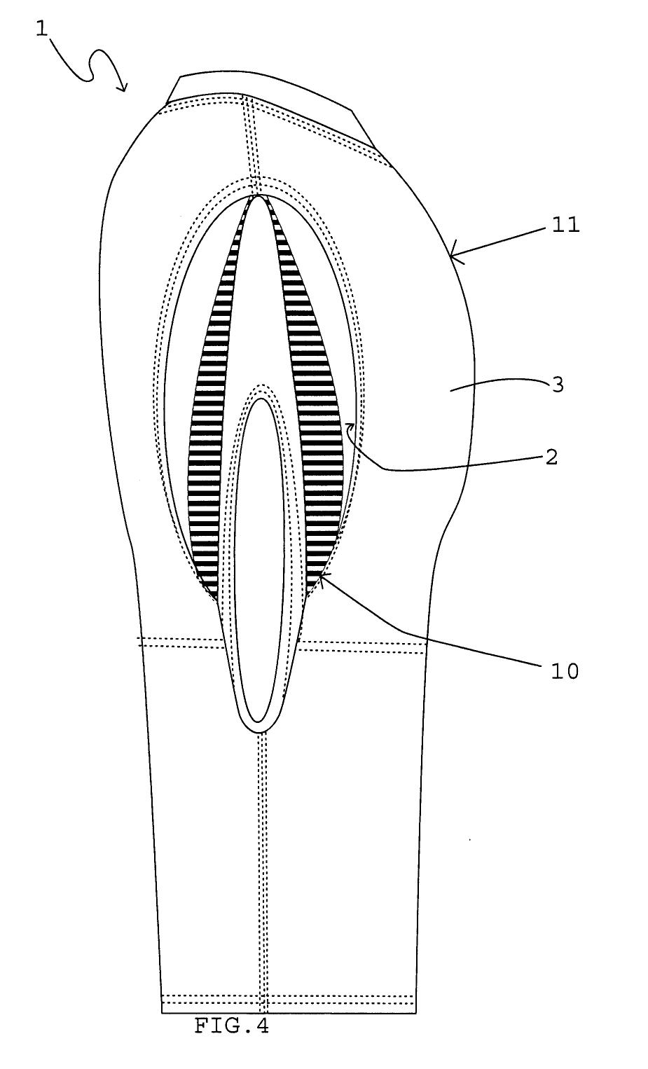

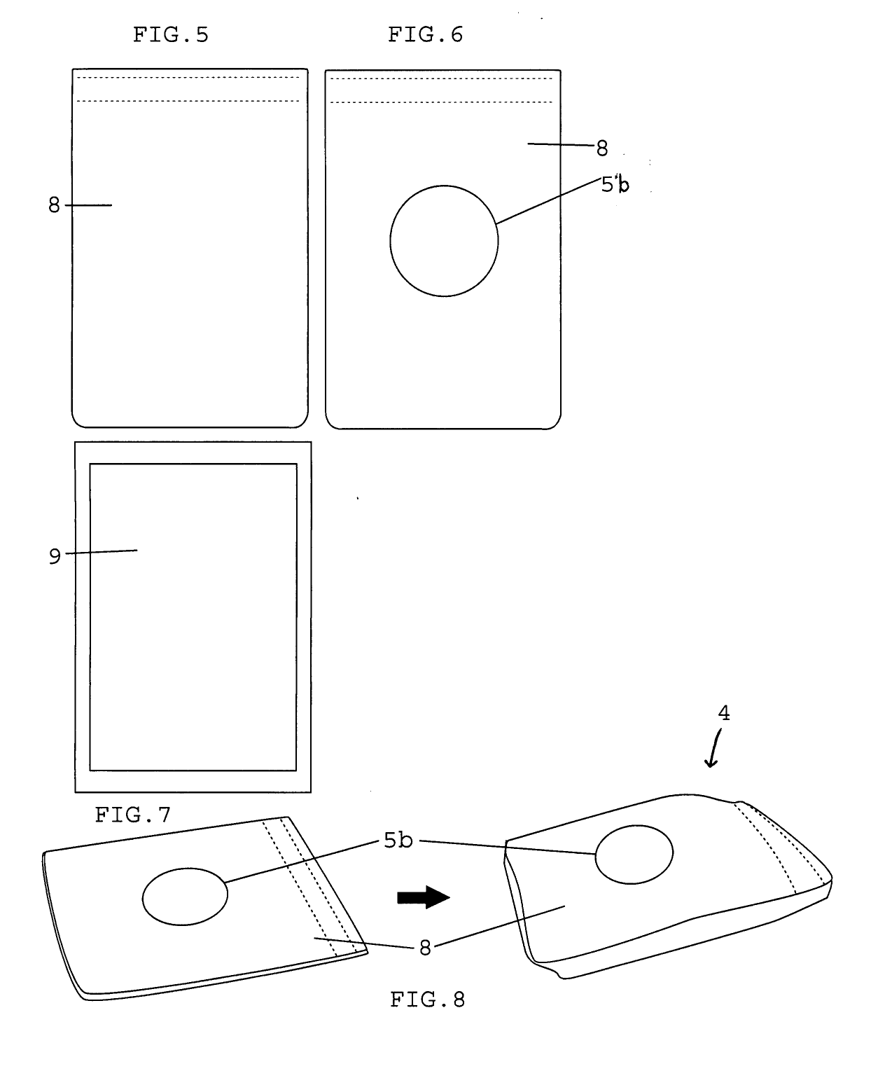

The thermal transfer element 4 is any object that is securable to the article of clothing 1 and is suitable for transferring heat. In some embodiments, the thermal transfer element 4 may simply comprise a thermal pack 9 if the thermal pack 9 is itself securable. However, in an exemplary embodiment of the invention, as best illustrated in FIGS. 5 to 8, the thermal transfer element 4 comprises a pouch 8 and thermal pack 9, the thermal pack 9 being contained in the pouch 8, the pouch 8 being removably and adjustably securable to the article of clothing 1. The pouch 8 may be made of any suitable material including natural and synthetic textiles, plastics, and rubbers, and it may be made of the same materials that the article of clothing 1 comprises. In an exemplary embodiment, the pouch 8 is approximately six inches by ten inches and has two sides that are sewn together to form a pouch. One side of the pouch 8 may be made of Thinsulate® laminated with variations of tricot, the other side of a mesh material which may be a blend of polyester and spandex. Thinsulate® is the registered trademark name of a material made by 3M Company (St. Paul, Minn.) that comprises thin and relatively dense polyolefin microfibers and polyester fibers. The two sides of the pouch 8 may be sewn together along three edges, leaving an opening on the fourth edge. Optionally the pouch 8 is closable on the fourth edge so that the thermal pack 9 may be completely enclosed in the pouch 8, as shown in FIG. 8. The means for clothing the pouch may be zipper, snap, button, or the like, but in an exemplary embodiment is a hook and loop fastener. The pouch 8 also comprises the second fastener 5B, meaning that this fastener may be either an integral part of the pouch 8 or attached thereto. In an exemplary embodiment, the second fastener 5B is hook material sewn to the Thinsulate-tricot side of the pouch 8.

The thermal pack 9 is any object suitable for transferring heat including, without limitation, heat packs and cold packs of the type commonly used as hot or cold compresses on injured or sore body parts, whether single-use or reusable. The thermal pack 9 may contain water, ice, gel, chemicals, or any other material, whether solid, liquid, or gas. Examples of some types of thermal packs include, without limitation, the Hot & Cold Flexible Gel Pack (3M, St. Paul, Minn.), Thermo-Pad Heat Packs (Hood Thermo-Pad, Summerlend, BC Canada), and the Hot/Cold Reusable Gel Pack (Accu-Therm, Taipei, Taiwan). The thermal pack 9 is brought to a desired temperature prior to attachment of the thermal transfer element 4 to the article of clothing 1 or, if possible, while the thermal transfer element 4 is attached. The thermal pack 9 may be heated or cooled by any suitable means including, without limitation, convection, conduction, radiation, electrical resistance, and chemical reaction. For example, the thermal pack 9 may be cooled by a refrigerator, freezer, ice water bath, or endothermic chemical reaction. The thermal pack 9 may be heated, for example, by an oven, microwave, hot water bath, or exothermic chemical reaction.

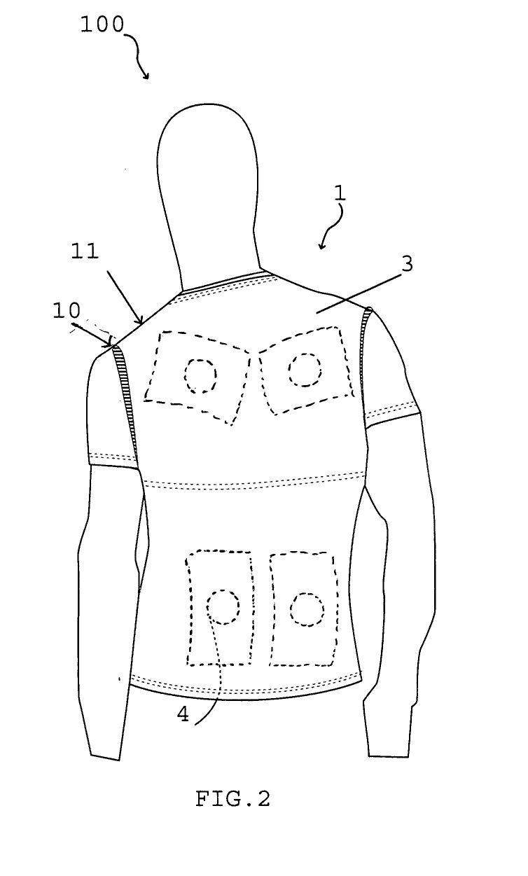

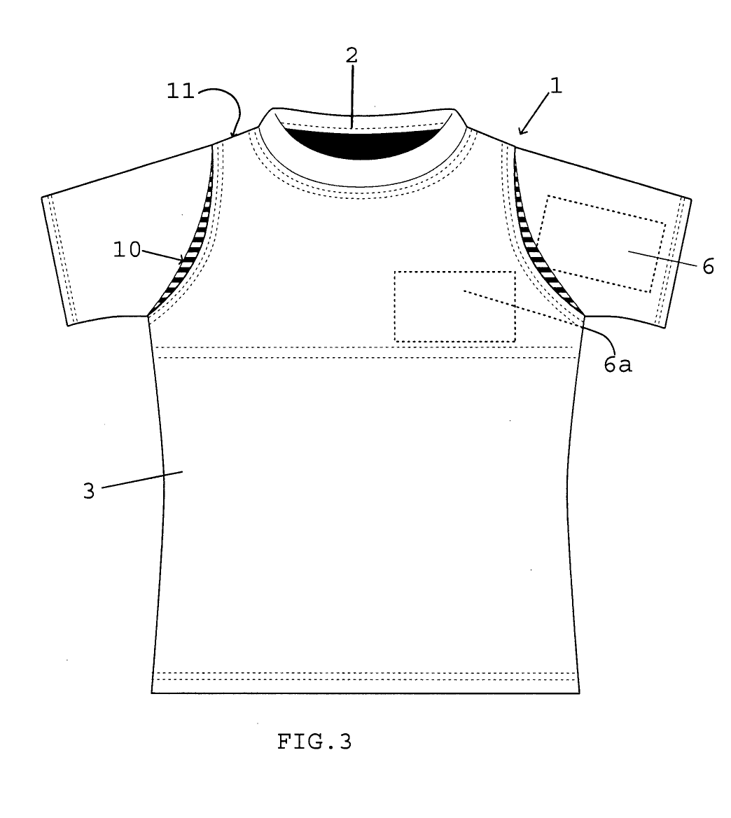

In an exemplary embodiment of the present invention, as best illustrated in FIGS. 1 and 2, the thermal transfer element 4 is adjustably secured to the interior surface 2 of the outer layer 11 of the article of clothing 1 by the engagement of first fastener 5A and second fastener 5B. As seen in FIG. 3, the thermal transfer element 4 may be removed from an initial location 6 by disengagement of the fasteners, and re-secured in a different location 6A by reengagement of the fasteners. The ability to disengage and reengage the fasteners 5 in a new location 6A makes the location 6 of the thermal transfer element 4 adjustable.

The first fastener 5A may be found anywhere on the article of clothing 1, including anywhere on any interior surface 2 or exterior surface 3 of any layer. In one embodiment, a surface of the article of clothing 1 itself comprises the first fastener 5A, such as where the material is brushed to create loop material suitable for a hook and loop fastener.

The second fastener 5B may be found anywhere on the thermal transfer element 4. In an embodiment where the thermal transfer element 4 comprises a thermal pack 9 contained in a pouch 8, the second fastener 5B may be found anywhere on the pouch 8. In an embodiment where the thermal transfer element 4 is a thermal pack 9, the second fastener 5B is found anywhere on the thermal pack 9. In an exemplary embodiment, as seen in FIGS. 6 and 8, the thermal pack 9 is contained in a pouch 8 having a second fastener 5B on its back side.

The first fastener 5A and the second fastener 5B are any type of complementary fasteners capable of repeatedly engaging and disengaging with each other, without significantly damaging either fastener or their ability to engage. It is not material which of the pair of complementary fasteners is the first fastener 5A or the second fastener 5B. For example, the first fastener 5A may be loop material and the second fastener 5B may be hook material. Likewise, the first fastener SA may be hook material and the second fastener 5B may be loop material. In an exemplary embodiment of the present invention, however, the first fastener SA is loop material that covers the interior surface 2 of the outer layer 11 of the article of clothing 1, and the second faster 5B is hook material that is sewn to the back of the pouch 8 of the thermal transfer element 4. Because in this embodiment the first fastener 5A is loop material covering the interior surface 2 of the outer layer 11, the thermal transfer element 4 is adjustable over the interior surface 2 by disengagement and reengagement of the fasteners 5.

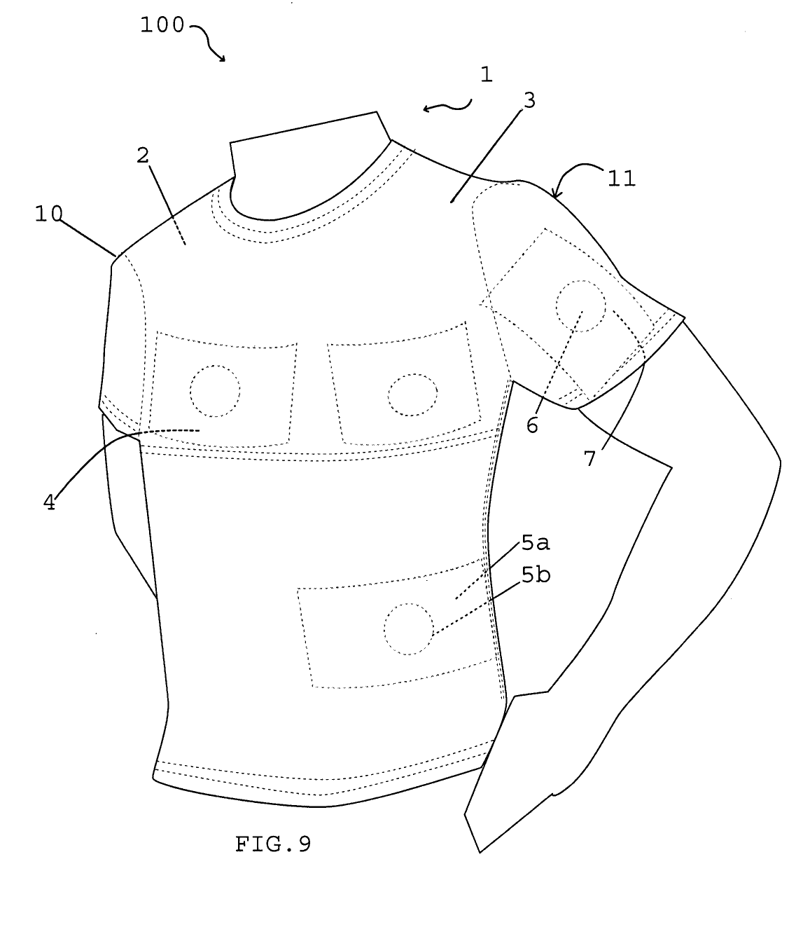

In another embodiment of the present invention, best illustrated in FIG. 9, a garment 100 comprises an article of clothing 1 having an interior surface 2 and an exterior surface 3, a thermal transfer element 4, a first fastener 5A and a second fastener 5B. Unlike the previous embodiment, the article of clothing 1, as shown in FIG. 10, does not comprise a first layer plus an outer layer, but instead comprises only a first layer 10. In this embodiment, the thermal transfer element 4 is secured to the interior surface 2 of the first layer 10 of the article of clothing 1. As seen in FIG. 9, the thermal transfer element 4 is drawn with dashed lines to indicate that the thermal transfer element 4 is attached inside the article of clothing 1. The article of clothing 1 may be made from a blend of nylon, polyester, and spandex, and the interior surface 2 of the first layer 10 may be brushed to create loop material suitable for a hook and loop fastener. In another embodiment, the thermal transfer element 4 may be adjustably secured to the exterior surface 3 of the first layer 10 of the article of clothing 1. In this embodiment, the exterior surface 3 of the first layer 10 may be brushed to create loop material suitable for a hook and loop fastener.

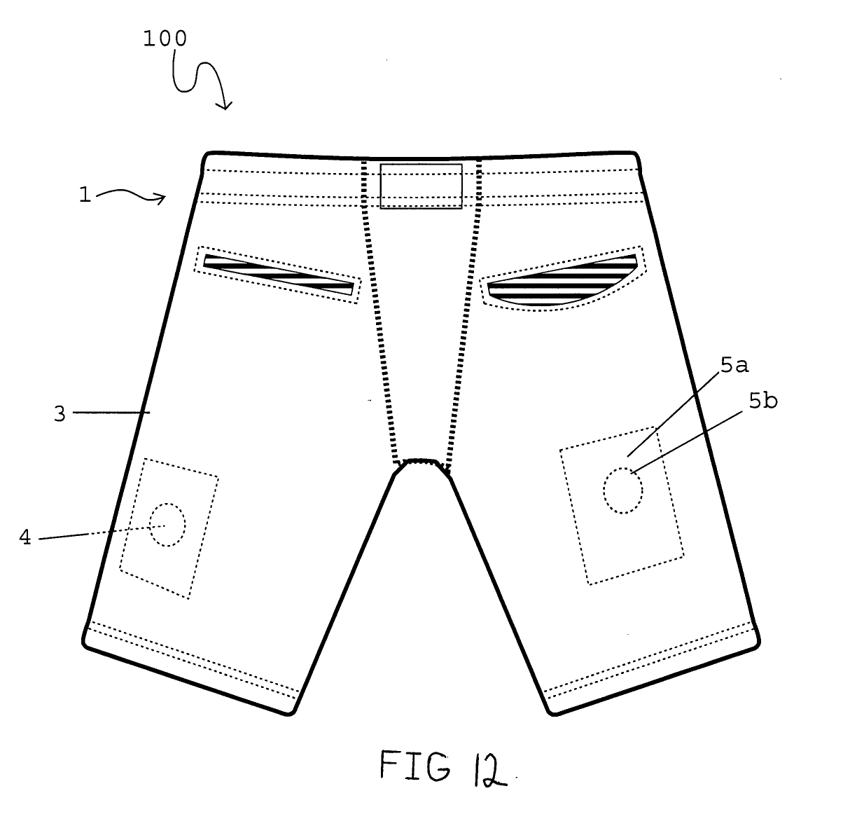

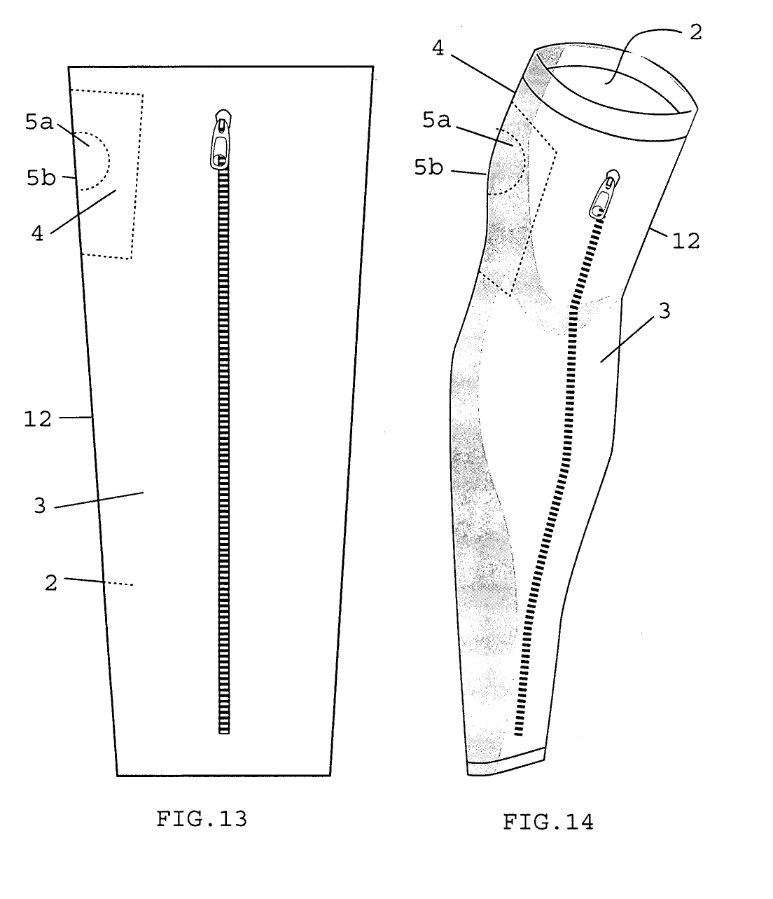

In another embodiment of the invention, the article of clothing 1 may comprise a detachable segment 12, such as a detachable sleeve for a shirt as illustrated in FIGS. 13 and 14. An article of clothing 1 may be worn with or without the detachable segment 12. Likewise, the detachable segment 12 may be worn without the rest of an article of clothing 1. The detachable segment 12 comprises the first fastener 5A, meaning that this fastener may be either an integral part of the detachable segment 12 or attached thereto.

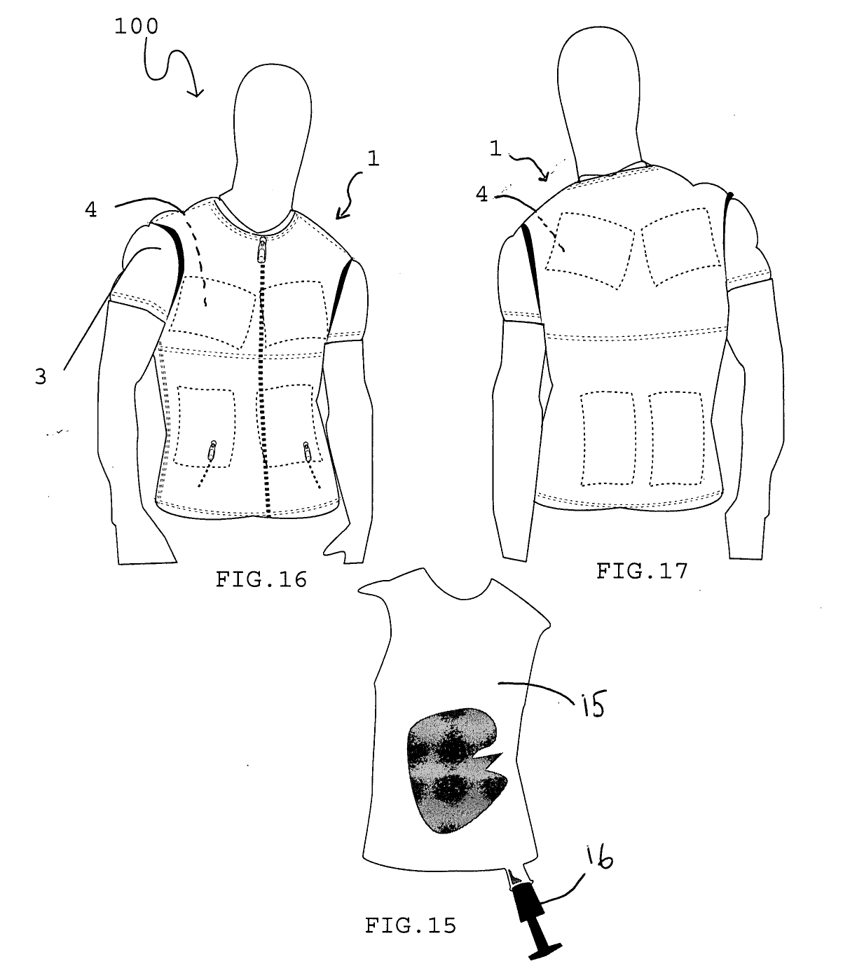

In other embodiments of the present invention, best illustrated in FIGS. 15 to 21, a thermal transfer element 4 is pressed against the body of the wearer by the article of clothing 1. In one embodiment, this pressure results from the snug fit of the article of clothing 1 against the body of the wearer. In one embodiment, shown in FIGS. 15 to 17, the pressure provided by the article of clothing 1 is augmented by inflation of an air bladder 15 contained inside the article of clothing 1. The air bladder 15 may be inflated by a one-way or sealable valve 16 for receiving pressurized air, such as by a person blowing into the valve. The air bladder 15 may also be inflated by a small pump built into the air bladder 15. After placing the thermal transfer element 4 in a desired location inside the article of clothing 1, the user inflates the bladder 15, thus providing increased pressure on the thermal transfer element 4 as the interior surface of the air bladder 15 expands toward the wearer.

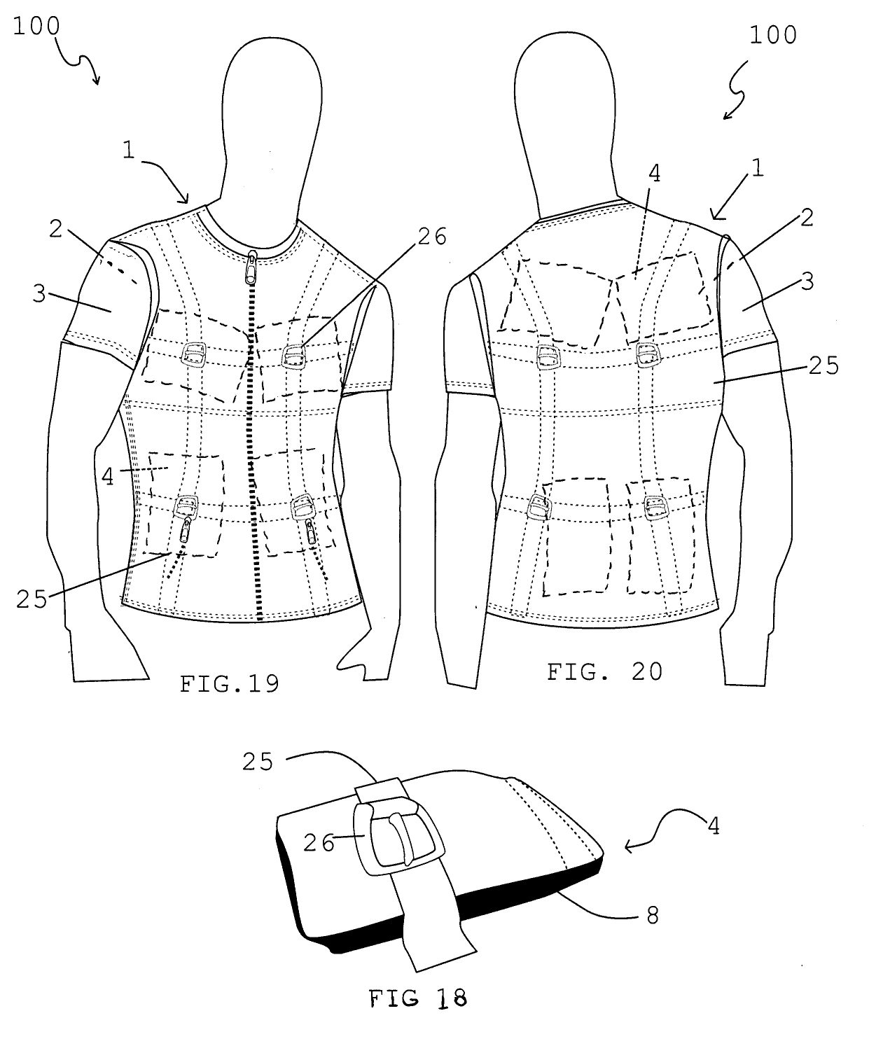

In another embodiment, as seen in FIGS. 18 to 20, the pressure against the thermal transfer element 4 is augmented by tightening straps 25 connected to buckles 26. After the user places the thermal transfer element 4 in a desired location, the user tightens the straps 25 using the buckles 26, thus providing increased pressure on the thermal transfer element 4. As shown in FIGS. 21 to 23, the tightentable straps may be cinch cords 35 that run throughout the article of clothing 1. After the user places the thermal transfer element 4 in a desired location, the user pulls on the cinch cord 35, thus providing increased pressure on the thermal transfer element 4.

A method of use according to an exemplary embodiment of the present invention is best understood with reference to FIGS. I to 8 and comprises: selecting a body part 7 to have a thermal pack 9 applied to it, putting on an article of clothing 1, selecting a location 6 on the article of clothing I that contacts the selected body part 7 and has a first fastener SA, bringing a thermal pack 9 to a desired temperature, engaging the first fastener 5A with the second fastener 5B to removably and adjustably secure the thermal pack 9 to the article of clothing 1, and permitting heat to transfer between the thermal pack 9 and the body part 7. The thermal pack 9 may be secured to the article of clothing 1 while the article of clothing 1 is being worn, while it is not being worn, or while it is partially being worn. Heat transfer continues until the thermal pack 9 reaches approximately the same temperature as the body part 7, at which point heat transfer begins to cease. In one embodiment, the second fastener 5B is affixed to the thermal pack 9. In an exemplary embodiment, the thermal pack 9 is enclosed in a pouch 8 with the second fastener 5B, preferably hook material, affixed to one side of the pouch 8. Furthermore, the above steps may be repeated in any order to attach a plurality of thermal packs 9 to a plurality of locations 6 contacting a plurality of body parts 7. These plurality of thermal packs 9 may be any combination of hot packs and cold packs.

Another method of use of the present invention, best understood with reference to FIGS. 1 to 8, is a method of heating or cooling the body of an athlete while the athlete is engaged in an athletic activity comprising selecting a body part 7 of an athlete to be heated or cooled, selecting a location 6 on the article of clothing 1 that contacts the selected body part 7 when the article of clothing 1 is worn and having a first fastener 5A, bringing a thermal pack 9 to a desired temperature, removably and adjustably securing the thermal pack 9 to the article of clothing 1 so that the thermal pack 9 is contacting the body part 7, and permitting heat to transfer between the body part 7 and the thermal pack 9 while the athlete is engaged in an athletic activity. Because the garment 100 holds the thermal pack 9 in place, the athlete's hands are free to participate in the athletic activity. In one embodiment, the second fastener 5B is affixed to the thermal pack 9. In an exemplary embodiment, the thermal pack 9 is enclosed in a pouch 8 with the second fastener 5B, preferably hook material, affixed to one side of the pouch 8. Furthermore, the above steps may be repeated in any order to attach a plurality of thermal packs 9 to a plurality of locations 6 contacting a plurality of body parts 7, so that more heat can be transferred to or from the athlete's body.

Another method of use of the present invention, best understood with reference to FIGS. 1 to 8, is a method of performing temperature enhanced physical therapy on a patient comprising selecting a body part 7 that will receive the therapy, putting the article of clothing 1 on the body of the patient, selecting a location 6 on the article of clothing 1 that contacts the selected body part 7 and has a first fastener 5A, bringing a thermal pack 9 to a desired temperature, removably and adjustably securing the thermal pack 9 to the article of clothing 1 by engaging a first fastener 5A and a second fastener 5B, and permitting heat to transfer between the thermal pack 9 and the selected body part 7 while the patient simultaneously receives physical therapy on the selected body part 7. In one embodiment, the second fastener 5B is affixed to the thermal pack 9. In an exemplary embodiment, the thermal pack 9 is enclosed in a pouch 8 with the second fastener 5B, preferably hook material, affixed to one side of the pouch 8. Furthermore, the above steps may be repeated in any order to attach a plurality of thermal packs 9 to a plurality of locations 6 contacting a plurality of body parts 7.

A further method of use of the present invention, also best understood by reference to FIGS. 1 to 8, is a method of warming the body of a person in a cold environment, comprising the steps of selecting a body part 7 to be warmed, putting an article of clothing 1 on the body of the person, selecting a location 6 on the article of clothing 1 that contacts the selected body part 7 when the garment is worn and having a first fastener 5A, heating a thermal pack 9 to a desired temperature, removably and adjustably securing the thermal pack 9 to the selected location 6 by engaging first fastener 5A and second fastener 5B, and permitting heat to transfer from the thermal pack 9 to the body part 7 in order to keep the wearer warm in a cold environment. In one embodiment, the second fastener 5B is affixed to the thermal pack 9. In an exemplary embodiment, the thermal pack 9 is enclosed in a pouch 8 with the second fastener 5B, preferably hook material, affixed to one side of the pouch 8. Furthermore, the above steps may be repeated in any order to attach a plurality of thermal packs 9 to a plurality of locations 6 contacting a plurality of body parts 7.

It should be understood that various alternative embodiments, not illustrated herein, are covered by the scope of the appended claims. For example, in one alternative the thermal transfer element may be adjustably secured to the exterior surface of the first layer, inside an outer layer. In this embodiment, the exterior surface of the first layer may be a brushed material suitable for use as loop material in a hook and loop fastener.

In another alternative embodiment of the present invention, the thermal transfer element may be adjustably secured to the interior surface of the first layer, whether or not there is also an outer layer. In this embodiment, the interior surface of the first layer may be a brushed material suitable for use as loop material in a hook and loop fastener.

In other alternative embodiments, the first fastener 5A and the second fastener 5B may be various different types of complementary fasteners other than hook and loop fasteners. For example, the fasteners may be metal or plastic snaps, hooks that engage with rivets, pairs of zipper chains engaged by a zipper, a button and buttonhole, or the like.

Various modifications and alterations of the invention will become apparent to those skilled in the art without departing from the spirit and scope of the invention, which is defined by the accompanying claims. For example, it should be noted that steps recited in any method claims below do no necessarily need to be performed in the order they are recited. For example, in certain embodiments, steps may be performed simultaneously. The accompanying claims should be constructed with these principles in mind.

Furthermore, any element in a claim that does not explicitly state “means for” performing a specified function or “step for” performing a specified function is not to be interpreted as a “means” or “step” clause as specified in 35 U.S.C. § 112, ¶ 6.

What is claimed is:

1. A garment for applying a thermal transfer element to a part of the body of a person, the garment comprising:
a. an article of clothing comprising a first layer of flexible material,
b. a thermal transfer element,
c. a first fastener located on the article of clothing, and
d. a second fastener located on the thermal transfer element,
whereby engagement of the first fastener and the second fastener removably and adjustably secures the thermal transfer element to the article of clothing.

2. The garment of claim 1 wherein the first fastener comprises loop material and the second fastener comprises hook material.

3. The garment of claim 1 wherein the first fastener comprises hook material and the second fastener comprises loop material.

4. The garment of claim 1 wherein the first fastener is located on the interior surface of the first layer of flexible material.

5. The garment of claim 1 wherein the first fastener is located on the exterior surface of the first layer of flexible material.

6. The garment of claim 1 wherein the interior surface of the first layer of flexible material comprises the first fastener.

7. The garment of claim 1 wherein the exterior surface of the first layer of flexible material comprises the first fastener.

8. The garment of claim 6 or claim 7 wherein the first fastener comprises loop material.

9. The garment of claim 6 or claim 7 wherein the first fastener comprises hook material.

10. The garment of claim 1 wherein the thermal transfer element comprises a thermal pack.

11. The garment of claim 1 wherein the thermal transfer element comprises:
a. a pouch, and
b. a thermal pack enclosed in the pouch,
wherein the second fastener is located on the pouch.

12. The garment of claim 11 wherein the pouch comprises the second fastener.

13. The garment of claim 1 wherein the article of clothing is selected from the group consisting of vest, short sleeve shirt, long sleeve shirt, sleeve, shorts, pants, legging, bodysuit, skirt, dress, gown, robe, glove, sock, and hat.

14. The garment of claim 1 wherein the article of clothing snugly conforms to the shape of a human body or a portion thereof, such that the article of clothing presses the thermal transfer element against the part of the body of a wearer.

15. The garment of claim 1 wherein the article of clothing further comprises an outer layer of flexible material.

16. The garment of claim 15 wherein the first fastener is located on the interior surface of the outer layer of flexible material.

17. The garment of claim 15 wherein the first fastener is located on the exterior surface of the outer layer of flexible material.

18. The garment of claim 15 wherein the interior surface of the outer layer of flexible material comprises the first fastener.

19. The garment of claim 15 wherein the exterior surface of the outer layer of flexible material comprises the first fastener.

20. The garment of claim 18 or claim 19 wherein the first fastener comprises loop material.

21. The garment of claim 18 or claim 19 wherein the first fastener comprises hook material.

22. The garment of claim 1 wherein the article of clothing further comprises a detachable segment, the article of clothing being wearable with or without the detachable segment.

23. The garment of claim 22 wherein the detachable segment comprises the first fastener.

24. A garment for applying a thermal transfer element to a part of the body of a person, the garment comprising:
a. an article of clothing comprising a layer of flexible material, and
b. a thermal transfer element located between the article of clothing and the part of the body of a person,
wherein the thermal transfer element is held in place by forces generated by the article of clothing.

25. The garment of claim 24 wherein the article of clothing snugly fits the wearer, whereby the snug fit of the article of clothing contributes to the forces holding the thermal transfer element in place.

26. The garment of claim 24 wherein the article of clothing further comprises a tightenable strap, whereby the forces holding the thermal transfer element in place are increased by tightening the strap and decreased by loosening the strap.

27. The garment of claim 24 wherein the article of clothing further comprises an inflatable air bladder, whereby the forces holding the thermal transfer element in place are increased by inflating the bladder and decreased by deflating the bladder.

28. The garment of claim 24 wherein the thermal transfer element further comprises an inflatable air bladder, whereby the forces holding the thermal transfer element in place are increased by inflating the bladder and decreased by deflating the bladder.

29. The garment of claim 1 or claim 24 wherein the article of clothing further comprises a moisture absorbent material.

30. The garment of claim 1 or claim 24 wherein the article of clothing further comprises a thermoconductive material.

31. The garment of claim 1 or claim 24 wherein the article of clothing further comprises thermoreflective material.

32. A method of heating or cooling a body part of a person, comprising:
a. selecting a body part with which to transfer heat,
b. bringing a thermal pack to a desired temperature,
c. putting an article of clothing on the body of a person, the article of clothing having a first fastener;
d. selecting a location on the article of clothing that contacts the selected body part when the article of clothing is worn,
e. removably and adjustably securing a thermal pack to the selected location by engaging the first fastener with a second fastener, and
f. permitting heat to transfer between the thermal pack and the selected body part.

33. The method of claim 32 further comprising placing the thermal pack inside a pouch having a second fastener.

34. The method of claim 32 wherein the first fastener is loop material and the second fastener is hook material.

35. The method of claim 32 wherein the first fastener is hook material and the second fastener is loop material.

36. A method of heating or cooling the a body part of an athlete while the athlete is engaged in an athletic activity, the method comprising:
a. selecting a body part to be heated or cooled,
b. putting an article of clothing on the body of an athlete, such that the selected body part is at least partially covered by the article of clothing;
c. selecting a location on the article of clothing that contacts the selected body part when the article of clothing is worn by the athlete,
d. bringing a thermal pack to a desired temperature,
e. removably and adjustably securing the thermal pack to the selected location, and
f. permitting heat the selected body part to be heated or cooled while the athlete is engaged in an athletic activity.

37. The method of claim 36 further comprising placing the thermal pack inside a pouch.

38. The method of claim 36 wherein the thermal pack is secured to the selected location using a hook and loop fastener.

39. A method of performing temperature enhanced physical therapy on a body part of a patient comprising:
a. selecting a body part of a patient to receive temperature enhanced physical therapy,
b. putting an article of clothing on the body of the patient,
c. selecting a location on the article of clothing that contacts the selected body part when the article of clothing is worn by the patient,
d. bringing a thermal pack to a desired temperature,
e. removably and adjustably securing the thermal pack to the selected location, and
f. permitting heat to transfer between the selected body part and the thermal pack while the patient receives physical therapy on the selected body part.

40. The method of claim 39 further comprising placing the thermal pack inside a pouch.

41. The method of claim 39 wherein the thermal pack is secured to the selected location using a hook and loop fastener.

42. A method of warming a body part of a person in a cold environment comprising the steps of:
a. selecting a body part to be warmed,
b. putting an article of clothing on the body of the person, the article of clothing having a first fastener;
c. selecting a location on the article of clothing that contacts the selected body part of the person when the article of clothing is worn,
d. bringing a thermal pack to a desired temperature,
e. removably and adjustably securing the thermal pack to the selected location by engaging the first fastener with a second fastener, and
f. permitting heat to transfer to the selected body part while the person is in a cold environment.

43. The method of claim 42 further comprising placing the thermal pack inside a pouch having a second fastener.

44. The method of claim 42 wherein the first fastener is loop material and the second fastener is hook material.

45. The method of claim 42 wherein the first fastener is hook material and the second fastener is loop material.

46. A method of pressing a thermal pack against a body part of a person to increase the rate of heat transfer between the thermal pack and the body part, the method comprising:
a. selecting a body part with which to transfer heat,
b. putting a snugly fitting article of clothing on the body of a person,
c. bringing a thermal pack to a desired temperature,
d. selecting a location on any surface of the article of clothing, other than the outermost surface, that contacts the selected body part when the article of clothing is worn;
e. securing the thermal pack to the selected location,
f. permitting the snug fit of the article of clothing to press the thermal pack against the selected body part, and
g. permitting heat to transfer between the thermal pack and the selected body part.

47. The method of claim 46 further comprising placing the thermal pack inside a pouch.

48. The method of claim 46 wherein the thermal pack is secured to the selected location using a hook and loop fastener.

49. A method of simultaneously heating one body part while cooling another body part, comprising:
a. selecting a first body part to heat,
b. selecting a second body part to cool,
c. heating a first thermal pack,
d. cooling a second thermal pack,
e. putting an article of clothing on the body of a person, the article of clothing having a first fastener and a second fastener;
f. selecting a first location on the article of clothing that contacts the selected first body part to be heated when the article of clothing is worn,
g. selecting a second location on the article of clothing that contacts the selected second body part to be cooled when the article of clothing is worn,
h. removably and adjustably securing the first thermal pack to the selected first location by engaging the first fastener with a third fastener,
i. removably and adjustably securing the second thermal pack to the selected second location by engaging the second fastener with a fourth fastener, and
j. permitting heat to transfer between the thermal packs and the selected body parts.

50. The method of claim 49 further comprising:
a. placing the first thermal pack in a pouch having a third fastener, and
b. placing the second thermal pack in a pouch having a fourth fastener.

51. The method of claim 49 wherein the first and second fasteners are loop material and the third and fourth fasteners are hook material.

52. The method of claim 49 wherein the first and second fasteners are hook material and the third and fourth fasteners are loop material.
